(19) United States
(12) Patent Application Publication
(10) Pub. No.: US 2020/0397455 A1
(43) Pub. Date: Dec. 24, 2020

(54) TENACULUM HAVING A FINGER LOOP

(71) Applicant: COVIDIEN LP, MANSFIELD, MA (US)

(72) Inventors: JACOB C. BARIL, NORWALK, CT (US); MATTHEW A. DININO, NEWINGTON, CT (US); SAUMYA BANERJEE, HAMDEN, CT (US); ROY J. PILLETERE, NORTH HAVEN, CT (US); JUSTIN J. THOMAS, NEW HAVEN, CT (US); MICHAEL N. BLANEY, NEW HAVEN, CT (US); NICOLETTE R. LAPIERRE, WINDSOR LOCKS, CT (US)

(21) Appl. No.: 16/447,057

(22) Filed: Jun. 20, 2019

(51) Int. Cl. A61B 17/28

(52) U.S. Cl. CPC A61B 17/2841; A61B 17/2816; A61B 2017/00438; A61B 17/2833; A61B 17/282

(57) ABSTRACT

A tenaculum includes a first handle and a second handle pivotally coupled to each other about a pivot pin. The first handle includes a palm grip configured to support a palm of a user's hand. The second handle includes a loop defined therein configured to support a user's digit to facilitate pivoting the first handle toward the second handle. A first grasper arm extends from the first handle. A second grasper arm extends from the second handle. The first and second grasper arms are configured to grasp tissue. A first ratchet extension extends from the first handle towards the second handle and a second ratchet extension extends from the second handle towards the first handle. The first ratchet extension is configured to selectively engaged the second ratchet extension upon movement of the first and second handles towards one another to engage tissue between the first and second grasper arms.

### BACKGROUND

### Technical Field

The present disclosure relates to surgical instruments and, more particularly, to a tenaculum having a finger loop.

### Background of Related Art

Many endoscopic surgical procedures require cutting or ligating blood vessels or vascular tissue. Due to the inherent spatial considerations of the surgical cavity, surgeons often have difficulty suturing vessels or performing other traditional methods of controlling bleeding, e.g., clamping and/or tying-off transected blood vessels. By utilizing an endoscopic electrosurgical forceps, a surgeon can either cauterize, coagulate/desiccate and/or simply reduce or slow bleeding simply by controlling the intensity, frequency and duration of the electrosurgical energy applied through the jaw members to the tissue. Most small blood vessels, i.e., in the range below two millimeters in diameter, can often be closed using standard electrosurgical instruments and techniques. However, if a larger vessel is ligated, or a relatively large tissue specimen is to be removed, it may be necessary for the surgeon to convert the endoscopic procedure into an open-surgical procedure, or to plan and carry out an open-surgical procedure.

A tenaculum is a plier-like instrument which relies on mechanical action between its jaws to grasp, clamp and constrict vessels or tissue. So-called “open forceps” are commonly used in open surgical procedures whereas “endoscopic forceps” or “laparoscopic forceps” are, as the name implies, used for less invasive endoscopic surgical procedures. Electrosurgical forceps (open or endoscopic) utilize both mechanical clamping action and electrical energy to effect hemostasis by heating tissue and blood vessels to coagulate and/or cauterize tissue. A tenaculum uses mechanical action between its jaws to constrict vessels and is commonly used in open surgical procedures to grasp, dissect and/or clamp tissue.

### SUMMARY

A tenaculum in accordance with aspects of the present disclosure includes a first handle and a second handle. The first and second handles are pivotally coupled to each other about a pivot pin. The first handle includes a palm grip configured to support a palm of a user's hand. The second handle includes a loop defined therein configured to support a user's digit to facilitate pivoting the first handle toward the second handle. A first grasper arm extends from the first handle. A second grasper arm extends from the second handle. The first and second grasper arms are configured to grasp tissue upon movement of the first and second handles toward each other. A first ratchet extension extends from the first handle towards the second handle and a second ratchet extension extends from the second handle towards the first handle. The first ratchet extension is configured to selectively engaged the second ratchet extension upon movement of the first and second handles towards one another to engage tissue between the first and second grasper arms.

In aspects according to the present disclosure, the loop of the second handle forms a closed loop configured to surround the user's digit along a plane extending between the first handle and the second handle. The loop includes a substantially straight portion having a first end and a second end. A curved portion connects the first and the second ends of the substantially straight portion to each other to surround the user's digit along the plane extending between the first handle and the second handle.

In aspects according to the present disclosure, the first grasper arm and the second grasper arm each include at least one tooth extending therefrom configured to facilitate engagement of tissue therebetween. The at least one tooth of the first grasper arm projects toward the at least one tooth of the second grasper arm.

In aspects according to the present disclosure, the first handle or the second handle is coated with an insulative material.

In aspects according to the present disclosure, the first grasper arm or the second grasper arm is coated with a conductive material.

In aspects according to the present disclosure, the first ratchet extension includes a plurality of teeth and the second ratchet extension includes a plurality of teeth. The first and second plurality of teeth are configured to selectively engage one another to secure tissue between the first grasper arm and the second grasper arm.

In aspects according to the present disclosure, the first handle includes a flange extending away from the second handle.

### BRIEF DESCRIPTION OF THE DRAWINGS

The accompanying drawings, which are incorporated in and constitute a part of this specification, illustrate embodiments of the present disclosure and, together with the detailed description below, serve to further explain the present disclosure, in which:

FIG. 1 is a perspective view of a tenaculum according to the present disclosure with first and second graspers disposed in an open position;

FIG. 2 is a top view of a tenaculum according to the present disclosure with first and second graspers disposed in an open position;

FIG. 3 is a top view of a tenaculum according to the present disclosure with first and second graspers disposed in an open position and illustrating a closing direction of the first and second graspers; and

FIG. 4 is an enlarged perspective view of a first ratchet extension engaged with a second ratchet extension according to the present disclosure.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

“About” or “approximately” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings.

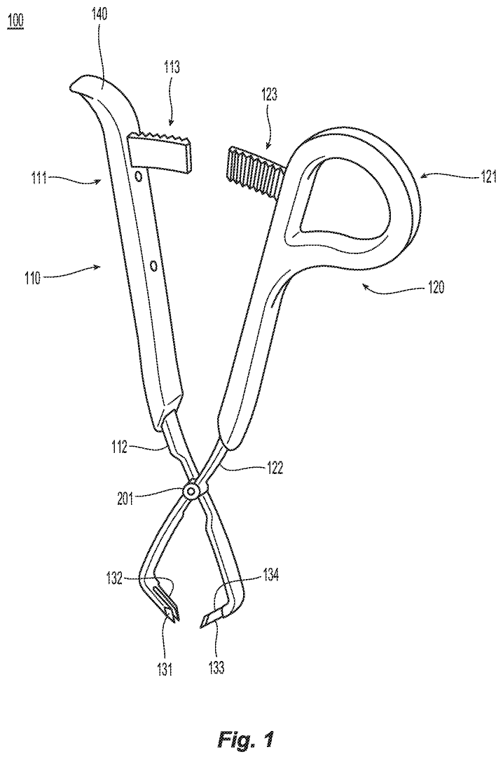

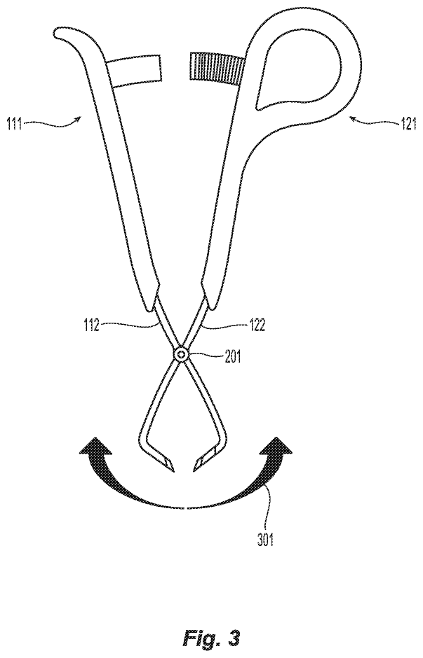

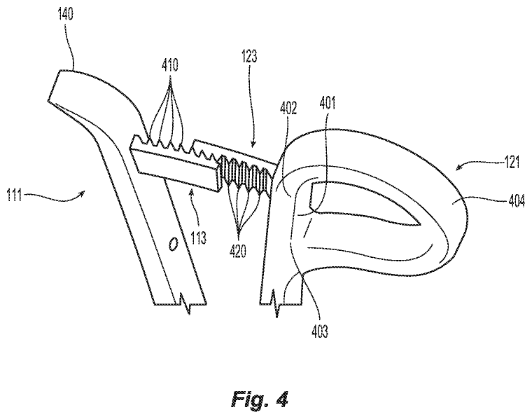

Referring to FIGS. 1 to 4, a tenaculum 100 in accordance with aspects of the present disclosure includes a first handle 110 and a second handle 120. The first and second handles 110 and 120 are pivotally coupled to each other about a pivot pin 201. The pivot pin 201 allows the first and second handles 110 and 120 to pivot about each other along a direction 301 (see, e.g., FIG. 3). The first handle 110 includes a palm grip 111 configured to support a palm of a user's hand. The second handle 120 includes a loop 121 defined therein configured to support a user's digit to facilitate pivoting the first handle 110 toward the second handle 120. A first grasper arm 112 extends from the first handle 110. A second grasper arm 122 extends from the second handle 120. The first and second grasper arms 112 and 122 are configured to grasp tissue upon movement of the first and second handles 110 and 120 toward each other. The tissue may be treatment tissue (e.g., tissue of a treatment organ or an entire organ) that is removed during a surgical procedure. The surgical procedure may be a laparoscopic or open surgical procedure; however, exemplary embodiments of the present disclosure are not limited thereto. The treatment tissue is pinched or gripped between a least some of the respective teeth (e.g., 131, 132, 133 and/or 134) extending from the first or second grasper arms 112 and 122.

A first ratchet extension 113 extends from the first handle 110 towards the second handle 120 and a second ratchet extension 123 extends from the second handle 120 towards the first handle 110. The first ratchet extension 113 is configured to selectively engaged the second ratchet extension 123 upon movement of the first and second handles 110 and 120 towards one another to engage tissue between the first and second grasper arms 112 and 122.

The first ratchet extension 113 includes a first plurality of teeth 410 and the second ratchet extension 123 includes a second plurality of teeth 420. The first and second plurality of teeth 410 and 420 are configured to selectively engage one another to secure tissue between the first grasper arm 112 and the second grasper arm 122. The combination of the palm grip 111 and the loop 121 allows an increased amount of pressure to be applied to pull the loop 121 relatively closer to the palm grip 111 compared with a conventional tenaculum. The increased pressure is maintained by the teeth 410 and 420 of the first and second ratchet extensions 113 and 123 that are removeably engaged with each other. Thus, the increased pressure is applied to the treatment tissue and the treatment tissue may be more easily removed as compared with a conventional tenaculum.

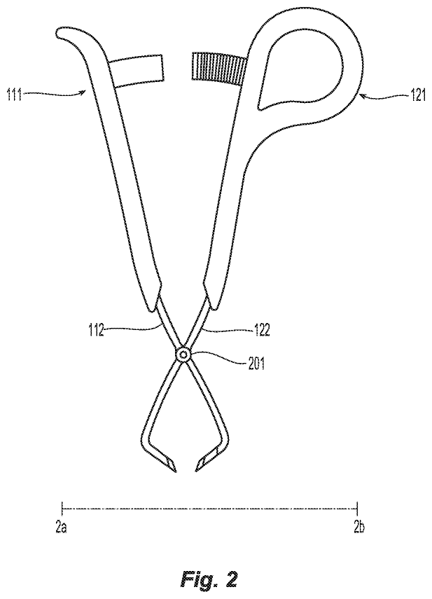

The loop 121 of the second handle 120 forms a closed loop configured to surround the user's digit along a plane (see, e.g., plane 2a-2b in FIG. 2) extending between the first handle 110 and the second handle 120. The loop 121 includes a substantially straight portion 401 having a first end 402 and a second end 403. A curved portion 404 connects the first and the second ends 402 and 403 of the substantially straight portion 401 to each other to surround the user's digit along the plane extending between the first handle 110 and the second handle 120.

The first grasper arm 112 and the second grasper arm 122 each include one or more teeth (e.g., teeth 131, 132 and teeth 133, 134, respectively) extending therefrom. The teeth, e.g. teeth 131, 133 are configured to facilitate engagement of tissue therebetween. The teeth, e.g., tooth 133 of the first grasper arm 112 projects toward the teeth, e.g., tooth 131, of the second grasper arm 122.

The first handle 110 or the second handle 120 is coated with an insulative material. In aspects according to the present disclosure, the first grasper arm 112 or the second grasper arm 122 is coated with a conductive material. Thus, electrosurgical energy may be applied to the first or second grasper arms 112 and/or 122 without exposing a user to the electrosurgical energy.

The first handle 110 includes a flange 140 extending away from the second handle 120. The flange 140 improves a user's grip and prevents a user's hand from sliding toward a proximal end of the first handle 110.

The tenaculum 100 according to the present disclosure is able to sit in the palm of a user's hand with the loop 121 engaged by the user's index finger (e.g., rather than user's other e.g., middle, finger). Thus, an increased amount of force may be applied to tissue through teeth 131-134, thus facilitating removal of the desired tissue or organ.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A tenaculum, comprising:
a first handle and a second handle, the first and second handles pivotally coupled to each other about a pivot pin, the first handle including a palm grip configured to support a palm of a user's hand, and the second handle including a loop defined therein configured to support a user's digit to facilitate pivoting the first handle toward the second handle;
a first grasper arm extending from the first handle, and a second grasper arm extending from the second handle, the first and second grasper arms configured to grasp tissue upon movement of the first and second handles toward each other; and
a first ratchet extension extending from the first handle towards the second handle and a second ratchet extension extending form the second handle towards the first handle, the first ratchet extension configured to selectively engaged the second ratchet extension upon movement of the first and second handles towards one another to engage tissue between the first and second grasper arms.

2. The tenaculum of claim 1, wherein the loop of the second handle forms a closed loop configured to surround the user's digit along a plane extending between the first handle and the second handle.

3. The tenaculum of claim 2, wherein the loop includes a substantially straight portion having a first end and a second end, a curved portion connecting the first and the second ends of the substantially straight portion to each other to surround the user's digit along the plane extending between the first handle and the second handle.

4. The tenaculum of claim 1, wherein the first grasper arm and the second grasper arm each include at least one tooth extending therefrom configured to facilitate engagement of tissue therebetween.

5. The tenaculum of claim 4, wherein the at least one tooth of the first grasper arm projects toward the at least one tooth of the second grasper arm.

6. The tenaculum of claim 1, wherein the first handle or the second handle is coated with an insulative material.

7. The tenaculum of claim 1, wherein the first grasper arm or the second grasper arm is coated with a conductive material.

8. The tenaculum of claim 7, wherein the first handle or the second handle is coated with an insulative material.

9. The tenaculum of claim 1, wherein the first ratchet extension includes a plurality of teeth and the second ratchet extension includes a plurality of teeth, the first and second plurality of teeth configured to selectively engage one another to secure tissue between the first grasper arm and the second grasper arm.

10. The tenaculum of claim 1, wherein the first handle includes a flange extending away from the second handle.
